(19) United States
(12) Patent Application Publication
(10) Pub. No.: US 2025/0102426 A1
(43) Pub. Date: Mar. 27, 2025

(54) SYSTEM AND METHOD FOR RAPID DETERMINATION OF FREE SULFUR DIOXIDE CONCENTRATION IN A LIQUID

(71) Applicant: BARRELWISE TECHNOLOGIES LTD., Vancouver (CA)

(72) Inventors: Miayan YEREMI, Vancouver (CA); Hugo RODRIGUEZ MARTINEZ, Vancouver (CA); Adrien Mathias NOBLE, North Vancouver (CA); David E. SOMMER, Vancouver (CA)

(21) Appl. No.: 18/703,232

(22) PCT Filed: Oct. 12, 2022
(86) PCT No.: PCT/CA2022/051497

§ 371 Date: Apr. 19, 2024

Related U.S. Application Data
(60) Provisional application No. 63/270,706, filed on Oct. 22, 2021.

(51) Int. Cl. G01N 21/33; G01N 33/00; G01N 33/14

(52) U.S. Cl. CPC G01N 21/33; G01N 33/0042; G01N 33/14; G01N 2201/0625

(57) ABSTRACT

A method for rapid determination of a concentration of free sulfur dioxide (SO₂) in a liquid is disclosed. The method involves receiving a liquid sample in an enclosed volume and, while a temperature of the liquid sample remains below 35° C., conditioning the liquid sample to cause gasified SO₂ to accumulate in a headspace of the enclosed volume above a surface of the liquid sample. The method further involves directing UV light having a wavelength between 250 nm and 320 nm through a gaseous sample taken from the headspace, and measuring an attenuation of the UV light due to absorption within the gaseous sample The concentration of gaseous SO₂ within the gaseous sample is determined from the measured attenuation.

### RELATED APPLICATIONS

This application claims the benefit of provisional patent application 63/270,706 entitled “SYSTEM AND METHOD FOR RAPID DETERMINATION OF FREE SULFUR DIOXIDE CONCENTRATION IN A LIQUID”, filed on Oct. 22, 2021 and incorporated herein by reference in its entirety.

### BACKGROUND

### 1. Field

This disclosure relates generally to the determination of a free sulfur dioxide concentration in a liquid, and more particularly to a rapid determination of free sulfur dioxide concentration.

### 2. Description of Related Art

Sulfur dioxide (SO₂) concentration within a liquid is an important parameter in several industries. In the beverage industry and particularly for fermented beverages such as wine, beer, and cider, SO₂ may be considered to be desirable in limited concentrations. In its dissolved form, SO₂ has antimicrobial and antioxidant effects. Some beverages may also include SO₂ that is bound to other constituents of the beverage such as aldehydes or ketones. The bound SO₂ is effectively inactive and not considered to be part of the free SO₂ that acts to prevent spoilage. Sulfites are however also considered to be food allergens and in some people may cause breathing difficulty, sneezing, hives, migraine, or other problems. This has led to certain jurisdictions placing limitations on total SO₂ concentration in beverages such as wine. The concentration of SO₂ in a beverage may thus be an important parameter to maintain and/or monitor.

Other industries also have a need for SO₂ concentration measurement. For example, boilers for heating water may be susceptible to tube fouling and sulfites may be added to mitigate this problem. The sulfite concentration may thus need monitoring to ensure that an effective concentration is maintained. Sulfites may also be used in water treatment industries where it is necessary to measure and control sulfite levels.

There is thus a need for improved techniques for measuring SO₂ concentration.

### SUMMARY

In accordance with one disclosed aspect there is provided a method for rapid determination of a concentration of free sulfur dioxide (SO₂) in a liquid. The method involves receiving a liquid sample in an enclosed volume, the liquid sample including a quantity of the liquid, and a quantity of acid to promote gasification of SO₂ within the liquid. The method also involves, while a temperature of the liquid sample remains below 35° C., conditioning the liquid sample to cause gasified SO₂ to accumulate in a headspace of the enclosed volume above a surface of the liquid sample to provide a gaseous sample. The method further involves directing UV light through the gaseous sample, the UV light having a wavelength within a spectral range of between 250 nm and 320 nm. The method also involves measuring an attenuation of the UV light due to absorption within the gaseous sample and determining the concentration of gaseous SO₂ within the gaseous sample from the measured attenuation, the gaseous SO₂ concentration being indicative of the free SO₂ concentration within the liquid sample.

The liquid may include a beverage.

The liquid may include dissolved or entrained carbon dioxide (CO₂) and conditioning the liquid sample may involve causing at least some CO₂ within the liquid sample to accumulate in the headspace and determining the attenuation of the UV light may involve determining the attenuation of the UV light in the presence of CO₂ in the gaseous sample.

The method may involve causing the liquid sample to have a temperature within a temperature range of between 18° C. and 35° C.

Receiving the liquid sample may involve receiving the liquid sample at a temperature of below 18° C. Receiving the liquid sample may involve passing the liquid sample through a heat exchanger to heat the liquid sample to a temperature within the temperature range.

The method may involve drawing the gaseous sample from the headspace and delivering the gaseous sample to a flow cell and directing the UV light through the gaseous sample may involve directing UV light through the flow cell.

Conditioning the liquid sample may involve sparging the liquid sample by drawing the gaseous sample from the headspace and recirculating the gaseous sample back below the surface of the liquid sample to cause free SO₂ in the liquid sample to be released into the headspace.

Sparging the liquid sample may involve delivering the recirculated gaseous sample to the enclosed volume at a location proximate a lower end of the enclosed volume.

Delivering the recirculated gaseous sample may involve directing the recirculated gaseous sample generally downwardly in the enclosed volume and with a component directed toward a lateral wall of the enclosed volume.

Recirculating the gaseous sample may involve passing the gaseous sample through a flow cell having an optical path therethrough and directing UV light through a gaseous sample may involve directing UV light through the optical path of the flow cell.

Drawing the gaseous sample may involve drawing the gaseous sample from a location above and spaced apart from the surface of the liquid sample to reduce a likelihood of liquid or foam from the liquid sample entering the recirculated gaseous sample and reaching the flow cell.

The enclosed volume may involve a passage that extends the headspace of the enclosed volume upwardly away from the surface of the liquid sample and drawing the gaseous sample may involve drawing the gaseous sample from a location proximate an upper end of the passage.

Drawing the gaseous sample may involve drawing the gaseous sample from the passage in a lateral direction.

Receiving the liquid sample may involve causing a liquid dosage system to draw the quantity of liquid from a container, and wherein a first portion of the quantity of liquid is delivered to the enclosed volume by operating the liquid dosage system, and a second portion of the quantity of liquid is delivered to the enclosed volume by flushing the liquid dosage system using a pressurized fluid. The liquid dosage system may be a liquid dosage pump. The pressurized fluid may be a pressurized gas.

The method may involve opening a vent valve in fluid communication with the headspace of the enclosed volume while flushing the liquid dosage system to permit pressurized fluid to escape. The pressurized fluid may be a pressurized gas.

The enclosed volume may include a drain port sealed by a drain valve at the bottom of the enclosed volume and the method may further involve, after the concentration of gaseous SO₂ within the gaseous sample has been determined, causing the drain valve to open to permit the liquid sample to be drained from the enclosed volume.

The drain valve may have a frustoconical shape and may be received in a frustoconical valve seat at the bottom of the enclosed volume and the valve may be configured to open by lifting upwardly out of the valve seat such that when being drained the liquid sample is completely drained to the bottom of the enclosed volume.

The method may involve flushing the enclosed volume by delivering a fluid to the enclosed volume that causes any remnants of the liquid sample to be forced out of the drain.

Directing UV light may involve directing UV light produced by a UV light emitting diode through the gaseous sample.

Measuring the attenuation of the UV light may involve generating modulated UV light, generating a measurement signal in response to receiving the UV light at a photodetector after passing through the gaseous sample, processing the measurement signal to extract components that are synchronized with the modulated UV light to determine an attenuation of the UV light, and determining the attenuation by comparing the level of attenuated UV light with a level of the generated modulated UV light.

Generating modulated light may involve generating UV light that may be intensity modulated at a reference frequency and processing the measurement signal may involve processing the measurement signal to extract components that are synchronized with the reference frequency to determine the attenuation of the UV light.

The photodetector may include a silicon carbide photodetector that is responsive to wavelengths of UV light in a narrow band including the 280 nanometer wavelength of the UV light.

The method may involve converting the measurement signal produced by the photodetector into a digital representation for receipt by a processor circuit and processing the measurement signal may involve mathematically processing the measurement signal in the processor circuit.

The method may involve splitting the UV light into a first beam of UV light and a second beam of UV light and directing UV light through the gaseous sample may involve directing the first beam of UV light along a measurement optical path through the gaseous sample in a measurement channel and may further involve directing the second beam of UV light through a reference optical path in a reference channel to generate a reference signal for extracting the measurement signal from noise.

The method may involve balancing the measurement channel and the reference channel while no gaseous sample is present.

Determining the concentration of gaseous SO₂ may involve producing an output absorption signal by taking a ratio of the measurement signal values and the reference signal and determining the SO₂ concentration based on fitting an exponential function to the output absorption signal.

The quantity of acid may include a quantity of phosphoric acid to lower a pH of the liquid sample to below pH 3.

In accordance with another disclosed aspect there is provided a system for rapid determination of a concentration of free sulfur dioxide (SO₂) in a liquid. The system includes an enclosed volume operable to receive a liquid sample, the liquid sample including a quantity of the liquid, and a quantity of acid to promote gasification of SO₂ within the liquid. The system also includes a liquid sample conditioner operably configured to condition the liquid sample to cause gasified SO₂ to accumulate in a headspace of the enclosed volume above a surface of the liquid sample while a temperature of the liquid sample remains below 35° C. The system further includes a flow cell in fluid communication with the headspace of the enclosed volume, the flow cell being operable to receive a gaseous sample taken from the headspace of the enclosed volume, the flow cell having an optical path therethrough. The system also includes a UV light source having a wavelength within a spectral range of between 250 nm and 320 nm, the UV light source being disposed to direct UV light through the optical path of the flow cell, a photodetector disposed to receive UV light passing through the gaseous sample and to generate measurement signal representing an attenuation of the UV light due to absorption within the gaseous sample. The system further includes a processor operably configured to determine the concentration of gaseous SO₂ within the gaseous sample based on the attenuation signal, the gaseous SO₂ concentration being indicative of the free SO₂ concentration within the liquid sample.

The liquid may include a beverage.

Other aspects and features will become apparent to those ordinarily skilled in the art upon review of the following description of specific disclosed embodiments in conjunction with the accompanying figures.

### BRIEF DESCRIPTION OF THE DRAWINGS

In drawings which illustrate disclosed embodiments,

FIG. 1 is a block diagram of a system for rapid measurement of a free sulfur dioxide (SO₂) in a liquid according to a first disclosed embodiment;

FIG. 2A is a perspective view of a reactor vessel used in the system of FIG. 1;

FIG. 2B is an exploded view of a reactor vessel of FIG. 2A;

FIG. 2C is a cross sectional view of an enclosed volume of the reactor vessel taken along a line 2C-2C in FIG. 2A;

FIG. 2D is a cross sectional view of the enclosed volume of the reactor vessel taken along a line 2D-2D in FIG. 2A;

FIG. 3 is a schematic view of an optical subsystem and controller for implementing the system shown in FIG. 1;

FIG. 4 is a schematic view of a SO₂ monitoring system for a beverage including the system shown in FIG. 1;

FIG. 5A is a process flowchart showing blocks of code for directing the processor circuit of FIG. 4 to perform an initialization process;

FIG. 5B is a process flowchart showing blocks of code for directing the processor circuit of FIG. 4 to perform a SO₂ concentration measurement;

FIG. 5C is a process flowchart showing blocks of code for directing the processor circuit of FIG. 4 to perform a purge process;

FIG. 6 is a process flowchart showing blocks of code for directing the processor circuit of FIG. 4 to perform a channel balancing process;

FIG. 7 is a graphical depiction of signals generated by the system during a first time period t; of the measurement process of FIG. 5B;

FIG. 8 is a graphical depiction of signals generated by the system during a second time period tₘ of the measurement process of FIG. 5B; and

FIG. 9 is a process flowchart showing blocks of code for directing the processor circuit of FIG. 4 to implement a digital signal process in accordance with one disclosed embodiment.

### DETAILED DESCRIPTION

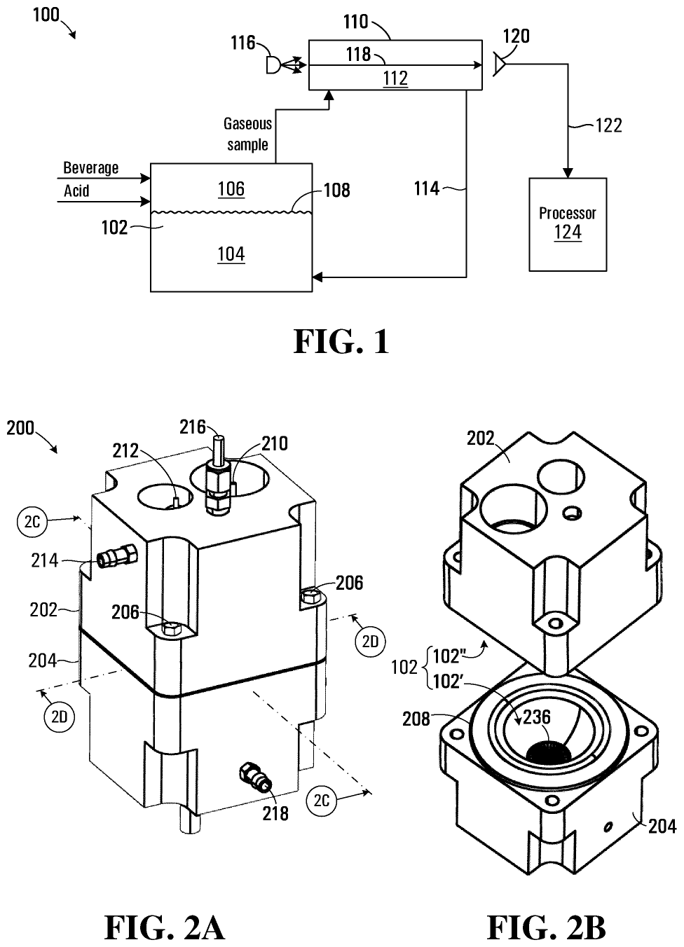

Referring to FIG. 1, a system for rapid measurement of a concentration of free sulfur dioxide (SO₂) in a liquid according to a first disclosed embodiment is shown generally at 100. The SO₂ measurement system 100 includes an enclosed volume 102 operable to receive a liquid sample 104. The liquid sample includes a quantity of the liquid and a quantity of acid. In the embodiment shown in FIG. 1, the quantity of liquid and quantity of acid are separately received in the enclosed volume 102 and thus mix within the enclosed volume to form the liquid sample 104. In other embodiments the liquid and acid may be mixed prior to being received in the enclosed volume 102. The acid is added to promote gasification of the free SO₂ within the liquid. In one embodiment phosphoric acid may be added in a quantity selected to lower a pH of the liquid sample to a pH below pH 3. The quantities of liquid and acid are selected such that a headspace 106 remains above a surface 108 of the liquid sample 104. In some embodiments, the size of the headspace 106 as a proportion of the liquid sample 104 may vary from the proportions shown in FIG. 1 and in particular the size of the headspace may occupy a significantly lesser proportion of the enclosed volume 102.

The system 100 also includes a flow cell 110 and a liquid sample conditioner. The flow cell 110 is in fluid communication with the headspace 106 and receives the gaseous sample taken from the headspace. The gaseous sample flows through an interior volume 112 of the flow cell. In this embodiment a return line 114 in fluid communication with the flow cell 110 acts as the liquid sample conditioner. That is, the return line 114 may be used to condition the liquid sample. The return line 114 recirculates the gaseous sample to cause sparging of the liquid sample 104. The sparging action of the recirculating gaseous sample in combination with the effect of the acid on hydrolysis accelerates gasification of the free SO₂ from the liquid sample 104 into the headspace 106. In other embodiments the liquid sample 104 may be sparged using a fluid other than the gaseous sample or the liquid sample may be otherwise conditioned by mechanical mixing, cavitation, vibration, or other means.

In this embodiment the liquid sample conditioner operates to condition the liquid sample 104 while a temperature of the liquid sample remains below 35° C. A substantial portion of the free SO₂ will be effectively gasified at temperatures below 35° C. while the bound SO₂ will substantially remain bound to other constituents. At temperatures above 35° C. the bound SO₂ may begin to be released and will add to the free SO₂ thus increasing the apparent free SO₂ level in the liquid. While in some cases it may be necessary to evaluate total SO₂ in a liquid, the disclosure herein relates to evaluation of only the free SO₂ concentration that is effective in preventing spoilage. As such, in this embodiment, the determination is made at temperatures below 35° C.

The flow cell 110 includes a measurement optical path 118 extending through the interior volume 112. The system 100 also includes a UV light source 116 having a wavelength within a spectral range of between 250 nm and 320 nm. The UV light source 116 is disposed to direct UV light through the optical path of the flow cell 110. The system 100 also includes a measurement photodetector 120 disposed to receive UV light passing through the gaseous sample within the interior volume 112 of the flow cell 110. The measurement photodetector 120 generates a measurement signal representing an attenuation of the UV light due to absorption within the gaseous sample.

The system 100 also includes a processor 124 operably configured to determine the concentration of gaseous SO₂ within the gaseous sample based on the attenuation signal. The gaseous SO₂ concentration is indicative of the free SO₂ concentration within the liquid sample. The processor 124 may be implemented using an analog electronic circuit, a digital electronic circuit, or a processor circuit.

SO₂ has a spectral absorption peak in the infrared wavelength region and also in the UV wavelength region around about 200 nm. However some liquids and particularly beverages such as wine may include varying concentrations of entrained or dissolved carbon-dioxide (CO₂). Some systems that have been previously implemented for measurement of SO₂ concentration in beverages use infrared wavelengths where CO₂ also exhibits significant spectral absorption. At these wavelengths it may thus be necessary to either eliminate CO₂ from the gaseous sample or attempt to separate the absorptive effects of CO₂ to determine the absorption due to SO₂ alone.

In this embodiment the spectral range of the UV light source 116 is specifically selected to be between 250 nm and 320 nm. Advantageously, relatively inexpensive UV sources and optical elements are available in this wavelength range. SO₂ also has a relatively prominent absorption peak in this wavelength range but CO₂ has effectively negligible absorption. This advantageously avoids the need to remove CO₂ from the liquid sample or otherwise compensate for the presence of CO₂ in the gaseous sample. In this embodiment the determination of SO₂ concentration may thus be made in the presence of CO₂ in the gaseous sample. The elimination of the need to remove CO₂ from the liquid sample also has the effect of reducing the time taken to obtain measurement results for the SO₂ concentration. During conditioning at least some CO₂ within the liquid sample thus accumulates in the headspace, and the attenuation of the UV light is determined in the presence of CO₂ in the gaseous sample.

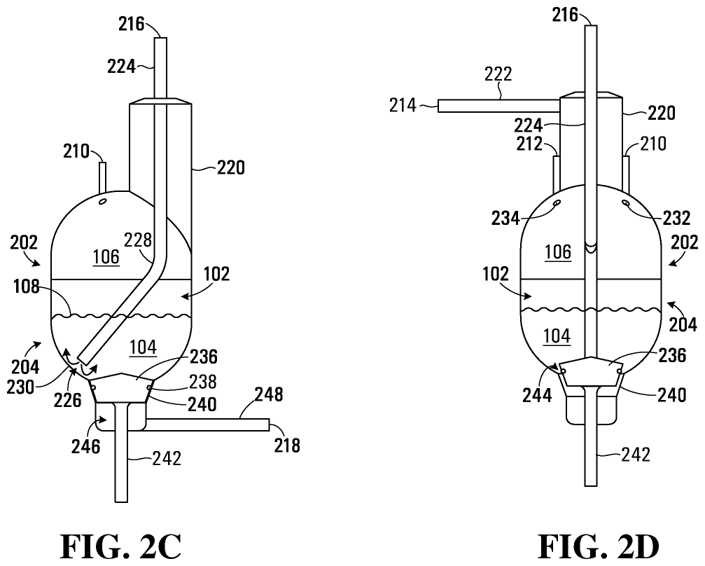

Referring to FIG. 2A, in one embodiment the enclosed volume 102 may be implemented using a two-part reactor vessel 200. The reactor vessel 200 includes an upper housing portion 202 and a lower housing portion 204. The upper housing portion 202 and the lower housing portion 204 are shown separated in FIG. 2B and the enclosed volume 102 includes a portion 102′ formed in the lower housing portion 204 and remaining portion 102″ formed within the upper housing portion 202. The lower housing portion 204 includes an o-ring seal 208 for sealing between the upper and lower housing portions when fastened together using bolts 206, as shown in FIG. 2A.

The upper housing portion 202 of the enclosed volume 102 includes a liquid inlet 210 for receiving the quantity of liquid and an acid inlet 212 for receiving the quantity of acid. The liquid inlet 210 and the acid inlet 212 are each connected via a conduit to respective openings 232 and 234 in the enclosed volume 102 for delivering the constituents of the liquid sample 104.

The upper housing portion 202 also includes a gas outlet 214 for receiving the gaseous sample taken from the headspace within the enclosed volume 102. The upper housing portion 202 further includes a gas inlet 216 for recirculating the gaseous sample through the liquid sample as described in more detail below. The lower housing portion 204 includes a drain outlet 218 for draining and flushing the liquid sample from the enclosed volume 102 when the SO₂ determination has been completed.

The enclosed volume 102 that is formed within the reactor vessel 200 is shown in first and second cross sectional views in FIGS. 2C and 2D taken along respective lines 20-2C and 2D-2D in FIG. 2A. In FIGS. 2C and 2D the enclosed volume 102 is shown schematically with portions of the reactor vessel 200 that define the enclosed volume and the interior conduits omitted for sake of illustration. Referring to FIG. 2C the enclosed volume 102 has a vertically elongated generally oval shape and that receives the liquid sample 104. In this embodiment, the reactor vessel includes a passage 220 that extends the headspace 106 upwardly within the enclosed volume 102 away from the surface 108 of the liquid sample 104. The gas outlet 214 is provided by a gas conduit 222 proximate the top of the passage 220. The gas conduit 222 also extends laterally from the passage 220 through the upper housing portion 202.

In this embodiment the gas inlet 216 is in fluid communication with a sparging tube 224 extending through the passage 220 and the headspace portion of the enclosed volume into the liquid sample 104. The sparging tube 224 has an opening 226 at a location proximate the bottom of the enclosed volume 102. A bend 228 in the tube 224 orients the opening such that the flow of the recirculated gaseous sample through the tube is directed generally downwardly and laterally at a sidewall 230 of the enclosed volume 102. The bend 228 in the tube 224 is selected to cause the opening 226 of the sparging tube 224 to be disposed as far away from the passage 220 and gas conduit 222 as possible.

The lower housing portion 204 includes a drain valve 236, which has a frustoconical shape and includes an o-ring seal 238. The drain valve 236 is received within a corresponding frustoconical shaped seat 240 (shown best in FIG. 2D) and includes a stem 242 that extends downwardly from a base of the enclosed volume 102. The stem 242 may be coupled to an actuator, such as a pneumatic actuator (not shown) configured to exert a downward force on the stem to cause the drain valve 236 to seal within the seat 240. As shown in FIG. 2B, the drain valve 236 thus forms a bottom surface of the enclosed volume 102 within the lower housing portion 204. When received in the seat 240 as shown in FIG. 2C, the valve 236 seals the enclosed volume 102 to contain the liquid sample 104. When the stem 242 is displaced upwardly by the actuator as shown in FIG. 2D, the drain valve 236 is unseated and provides an annular opening 244 for draining the liquid sample 104 from the enclosed volume 102 via a catchment volume 246 and through a drain conduit 248.

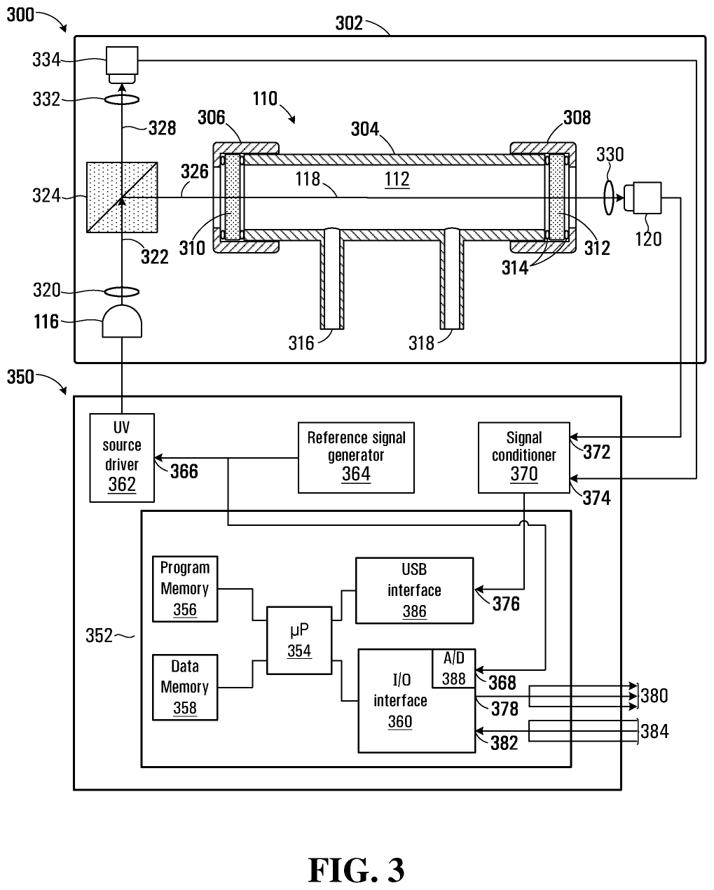

The flow cell 110, UV light source 116, measurement photodetector 120 and other optical elements of the system 100 form an optical subsystem shown in FIG. 3 at 300. The optical subsystem 300 may be mounted on an optical breadboard 302 or other mounting arrangement. In one embodiment the flow cell 110 may be implemented using a glass cell 304 having end caps 306 and 308. A suitable glass cell having threaded ends for receiving end caps is available from Thorlabs Inc. of New Jersey, United States. The end caps 306 and 308 accommodate respective optical windows 310 and 312, which provide the optical path 118 through the cell 304. The windows 310 and 312 are mounted between compressible seals 314 that seal off the interior volume 112 of the cell 304. The cell 304 also includes an inlet port 316 and an outlet port 318. The inlet port 316 receives the gaseous sample from the gas outlet 214 of the enclosed volume 102. The outlet port 318 recirculates the gaseous sample back to the gas inlet 216. In other embodiments the ports 316 and 318 may be reversed since the direction of flow of the gaseous sample through the interior volume 112 of the flow cell 110 may be in either direction. The components of the flow cell 110 may be implemented using one or more off the shelf components and/or customized parts. Using customized parts may improve packaging and integration with the surrounding optical systems.

In this embodiment the UV light source 116 is mounted to the optical breadboard 302 and may be implemented using a single color UV light emitting diode (UV-LED). In one embodiment the UV-LED may have a wavelength of 280 nm and a FWHM wavelength distribution width of about 12 nm. One or more lens elements 320 may be disposed to capture the UV radiation generated by the UV light source 116 and produce a UV light beam 322 that has a diameter selected to pass through the windows 310 and 312 without significant clipping. Other UV light sources such as a UV laser may be implemented in place of the UV-LED.

The optical subsystem 300 also includes a beamsplitter 324 that separates the UV light beam 322 into a measurement beam 326 and a reference beam 328. In one embodiment the beamsplitter 324 may be configured for a 50:50 split ratio such that the measurement beam 326 and the reference beam 328 have substantially similar intensity. In other embodiments the beamsplitter 324 may be configured for other than a 50:50 split ratio. The measurement beam 326 is directed through the cell 300 via the window 310 where it interacts with the gaseous sample in the interior volume 112 of the flow cell 110, before passing out of the cell via the window 312. The measurement photodetector 120 is disposed and aligned to receive the measurement beam 326 exiting the flow cell 110 via a one or more lenses 330 that collect and focus the measurement beam onto the measurement photodetector 120. The measurement beam 326 thus passes along the measurement optical path 118 through the interior volume 112 of the flow cell 110 that will receive the gaseous sample and forms a measurement channel of the optical subsystem 300. The reference beam 328 is directed through one or more lenses 332 that collect and focus the reference beam onto a reference photodetector 334. The reference beam 328 passes through a reference optical path aligned with the reference photodetector 334 but does not pass through the gaseous sample. The reference optical path and the reference photodetector 334 together form a reference channel of the optical subsystem 300.

In this embodiment the photodetectors 120 and 334 are implemented using Silicon carbide (SIC) photodetectors, which typically have a spectral response limited to wavelengths in the region of about 210 nm to 380 nm. SiC photodetectors are not sensitive to radiation outside this range, which allows the optical system 300 to be operated without additional filtering to prevent stray ambient light from influencing the signals detected by the photodetectors. In other embodiments the photodetectors 120 and 330 may be implemented using other types of photo-sensitive detectors.

The system 100 also includes a controller 350 for controlling operation of the system. In the embodiment shown the controller 350 includes a processor circuit 352. The processor circuit 352 includes a microprocessor 354, a program memory 356, a data memory 358, an Input/Output (I/O) interface 360, and a USB interface 386, all of which are in communication with the microprocessor 354. Program codes for directing the microprocessor 354 to carry out various functions are stored in the program memory 356, which may be implemented as a solid state memory, random access memory (RAM) and/or a hard disk drive (HDD), or a combination thereof. The I/O interface 360 may include an analog to digital signal converter 388 that is capable of receiving analog or digital input signals, converting analog input signals into digital representations, and generating output signals for controlling the system 100. The data memory 358 is used to store variables and data associated with operation of the system 100. In other embodiments (not shown), the controller 350 may be partly or fully implemented using a different processor circuit or using analog and/or digital elements in place of the processor circuit 352.

The controller 350 also includes a UV source driver 362, which generates a drive signal for powering the UV light source 116. The controller 350 also includes a reference frequency generator 364, which in this embodiment is configured to generate a sinusoidal reference frequency U_{REF} having a fixed frequency. In this embodiment the reference frequency U_{REF} has a frequency of about 1 kHz. The UV source driver 362 includes a modulation input 366 and is configured to modulate the drive signal based on the reference frequency U_{REF}.

The UV light produced by the UV light source 116 will thus have an intensity that is modulated at the reference frequency. The I/O interface 360 also includes an input 368 for receiving the reference signal, which is converted into a digital representation by the analog to digital signal converter 388 and is used for performing synchronous detection as described in more detail below.

In the embodiment shown the controller 350 also includes a signal conditioner 370, which includes a pair of inputs 372 and 374 for receiving respective signals from the measurement photodetector 120 and the reference photodetector 334. The signal conditioner receives raw signals from the photodetectors 120 and 334, conditions the signals, and converts the signals into digital representations thereof. The digital representations are provided to the processor circuit 352 via a data input 376 of the USB interface 386. In one embodiment the signal conditioner may be implemented using a two channel 24-Bit/192 kHz Interface, such as the UMC202HD audio interface manufactured by Behringer.

In this embodiment the I/O interface 360 includes one or more outputs 378 that provide a plurality of control signals 380 for controlling the system 100. For example, the one or more outputs 378 may be used to generate control signals 380 for controlling valves, actuators, and other elements as described in more detail later. The I/O interface 360 also includes one or more inputs 382, which may be used to receive input signals 384 such as status signals associated with valves, actuators, or other elements, or signals representing various operating parameters such as temperatures of fluids in various parts of the system 100.

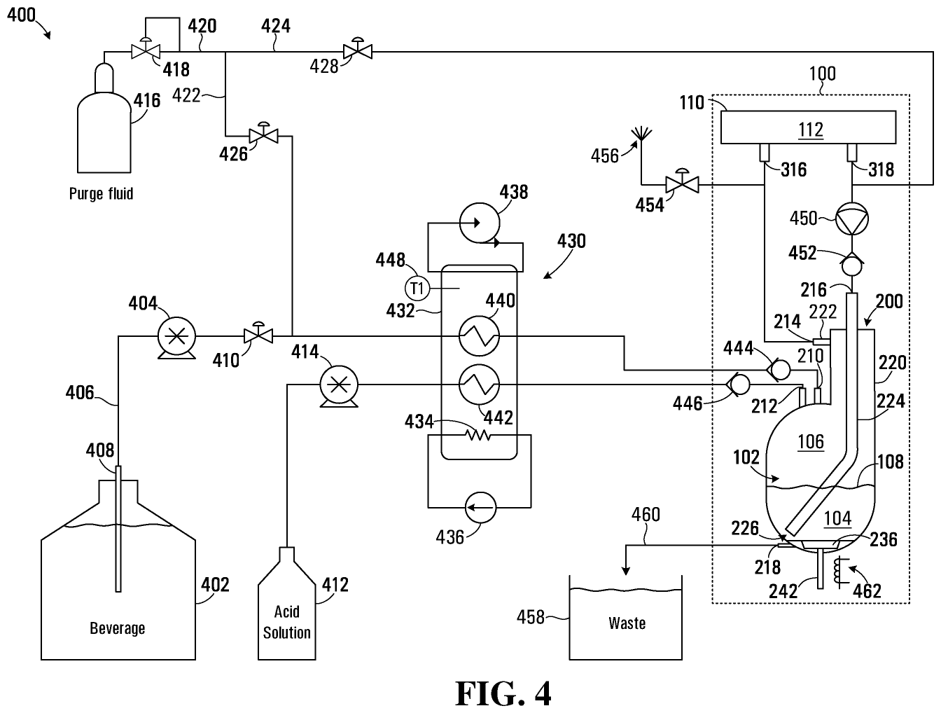

Referring to FIG. 4, a schematic diagram of a SO₂ monitoring system for a beverage is shown schematically at 400. The beverage monitoring system 400 includes the SO₂ measurement system 100 of FIG. 1 and further includes elements that interact with the SO₂ measurement system to sample a beverage, deliver and condition the liquid sample 104, and to flush the liquid sample once the measurement is completed. The beverage monitoring system 400 includes a plurality of fluid flow elements that may be controlled via the control signals 380 generated by the processor circuit 352. As disclosed above the SO₂ measurement system 100 may alternatively be implemented to perform other non-beverage SO₂ monitoring functions.

In this embodiment the beverage is held in a container 402, which may be a stainless steel tank, a concrete tank, a wooden barrel, or other beverage container. The monitoring system 400 also includes a beverage dosage pump 404, which is connected via a liquid sample line 406 to a dip tube 408 inserted into the container 402. The beverage dosage pump 404 is activated via one of the control signals 380 and is connected to a beverage valve 410, which is also actuated by one of the control signals 380. The acid solution is held in a container 412. Acid is drawn from the container 412 by an acid dosage pump 414. The dosage pumps may be implemented using a small peristaltic dosage pump that are actuated by the control signal 380 for a fixed period of time to deliver a desired quantity of beverage or acid. Alternatively, other pumps that are operable to deliver a precise volume of liquid such as piston or syringe pumps may be used in place of the peristaltic pump.

The monitoring system 400 also includes a purge fluid 416 for performing purging functions during and subsequent to measurement of SO₂ concentration. The purge fluid may be a pressurized gas supply such as nitrogen or air connected through a pressure regulator valve 418 to provide a regulated gas feed on a line 420. The pressure regulator valve 418 may be set to provide a fixed purge pressure. In the embodiment shown, the line 420 is split into two gas lines 422 and 424. The gas line 422 includes a push valve 426 and the gas line 424 includes a purge valve 428, which are respectively activated and de-activated via control signals 380 generated by the processor circuit 352 at the output 378 of the I/O interface 360. The line 422 is connected to the output of the beverage valve 410 and the function of this line is described in more detail below. In other embodiments the purge fluid may be a pressurized liquid such as water.

The monitoring system 400 also includes a heat exchanger 430. The heat exchanger 430 may be configured as a water bath 432 which is heated via a heater element 434 connected to a current source 436. In other embodiments a glycol based coolant may be used in place of water or the water may include an additive such as a corrosion inhibitor. In this embodiment the heat exchanger 430 also includes a circulation pump 438 that circulates the water within the water bath 432 to promote a substantially uniform temperature throughout the bath. The heat exchanger 430 further includes first and second heat exchanger coils 440 and 442, which may be implemented using coils of thermally conductive tubing immersed in the water bath 432. The outlet of the beverage valve 410 is connected through the first heat exchanger coil 440. The first heat exchanger coil 440 is connected via a check valve 444 to the liquid inlet 210 of the reactor vessel 200. The outlet of the acid dosage pump 414 is connected through the second heat exchanger coil 442. The second heat exchanger coil 442 is connected via a check valve 446 to the acid inlet 212 of the reactor vessel. The check valves 444 and 446 are located close to the respective liquid inlet 210 and acid inlet 212. The heat exchanger 430 also includes a temperature sensor 448 (T1) for sensing a temperature of the water in the water bath 432. The temperature sensor 448 produces a temperature signal that is received by the processor circuit 352 as one of the input signals 384 at the input 382 of the I/O interface 360.

In other embodiments, various other heat exchanger configurations such as a counter-flow heat exchanger or finned shell and tube heat exchangers may be implemented to provide packaging or performance improvements. A solid-to-liquid heat exchanger utilizing electric heating and cooling via a heating coil or a Peltier thermo-electric device may also be used to eliminate the need for a working fluid and to improve portability of the system.

The gas outlet 214 of the reactor vessel 200 is connected to the inlet port 316 of the flow cell 110 and the outlet port 318 is connected via a recirculation pump 450 and a check valve 452 to the gas inlet 216 of the reactor vessel 200. In one embodiment, the recirculation pump 450 may be implemented using a diaphragm pump actuated in response to one control signals 380 to cause flow of the gaseous sample through the pump.

The outlet port 318 of the flow cell 110 is also connected to the purge valve 428, while the inlet port 316 is connected via a vent valve 454 to a vent 456 that is open to the atmospheric environment. The vent valve 454 is configured to open in response to one of the control signals 380 generated by the processor circuit 352. The vent valve 454 when opened permits gas to escape through the vent 456 from the enclosed volume 102 and interior volume 112 of the flow cell 110 or permits air to enter the enclosed volume and interior volume.

The monitoring system 400 also includes a waste receptacle 458, which is in communication via a waste line 460 with the drain outlet 218 of the reactor vessel 200. The drain valve 236 is actuated to open and close by exerting a force on the stem 242 via an actuator 462 such as a solenoid or pneumatic actuator. The actuator 462 is controlled by one of the control signals 380 generated by the processor circuit 352. The location of the drain valve 236 and seat 240 at the bottom of the enclosed volume 102 ensures that the liquid sample is completely drained to the bottom of the enclosed volume.

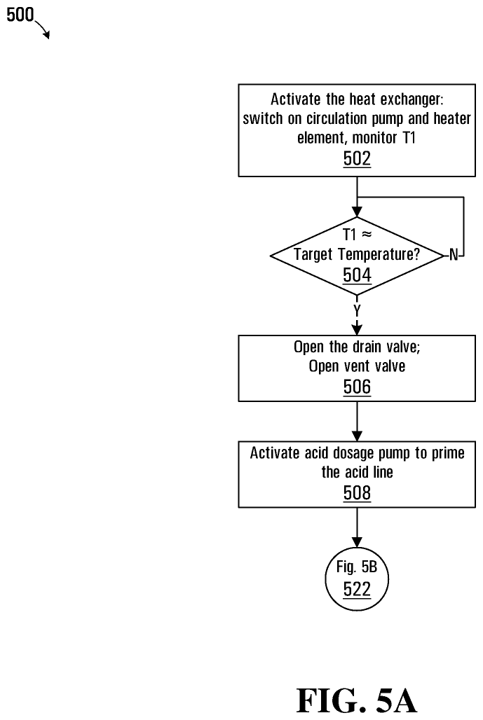

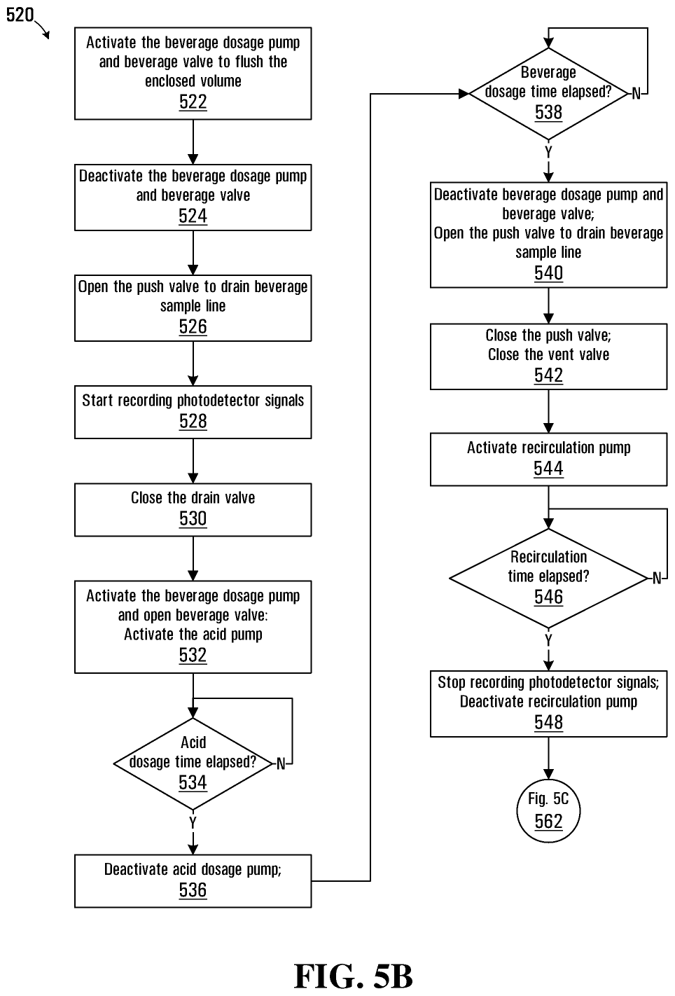

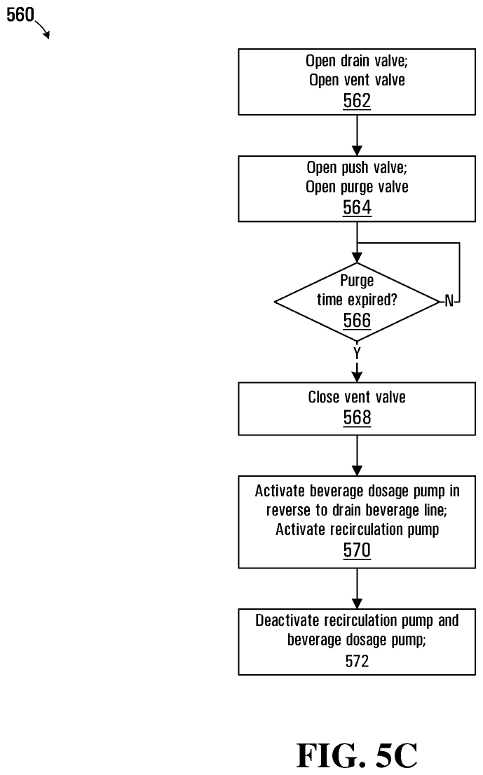

The operation of the beverage monitoring system 400 is described further with reference to flowcharts shown in FIGS. 5A, 5B and 5C that depict blocks of code for directing the processor circuit 352 to perform the SO₂ concentration measurement. The blocks generally represent codes that may be read from the program memory 356 for directing the microprocessor 354 to perform various functions related to the SO₂ measurement. The actual code to implement each block may be written in any suitable program language, such as Python, C, C++, C#, Java, and/or assembly code, for example.

Referring to FIG. 5A, an initialization process embodiment is shown generally at 500. The initialization process 500 may be performed when initially preparing the system for measurement but need not be repeated prior to measurements on each of a plurality of different beverage containers. Initially the enclosed volume 102 of the reactor vessel 200 would have been drained after a previous measurement and should be substantially empty except for some possible residual of liquid sample from the previous measurement. The water bath 432 of the heat exchanger 430 would also have been filled with water.

The initialization process begins at block 502, which directs the microprocessor 354 activate the heat exchanger 430. This involves directing the microprocessor 354 to generate the necessary control signals 380 to switch on the circulation pump 438 of the heat exchanger 430 and cause the current source 436 to deliver current to the heater element 434 for heating the water in the water bath 432. Block 502 also directs the microprocessor 354 to monitor the temperature signal T1 generated by the temperature sensor 448 by monitoring the applicable input signal 384.

The process 500 then continues at block 504, which directs the microprocessor 354 to determine whether the temperature T1 of the water bath 432 has reached a target temperature. In one embodiment an appropriate target temperature for determining SO₂ in a beverage may be in the range of 18° C. to 35° C. Beverages such as wine may ferment at temperatures above 20° C. but would be aged at a lower temperature, typically below 18° C. Depending on the point in the winemaking process, there may or may not be a need to heat the beverage prior to SO₂ measurement. As an example, the target temperature may be set to 25° C. and block 504 would then direct the microprocessor 354 to maintain the temperature of the water bath 432 at approximately 25° C., within an allowable variance range. If T1 is outside the variance range, block 504 directs the microprocessor 354 to repeat block 504. When T1 is inside the variance range, block 504 directs the microprocessor 354 to block 506. Block 506 may continue to be executed in parallel with other blocks of the process 500 to maintain the temperature T1 within the allowable variance range.

Block 506 then directs the microprocessor 354 to generate a control signal 380 that causes the drain valve 236 to be opened. Block 506 also directs the microprocessor 354 to generate a control signal 380 that causes the vent valve 454 to be opened. The process then continues at block 508, which directs the microprocessor 354 to generate a control signal 380 to activate the acid dosage pump 414 for priming the acid line connected to the acid inlet 212 of the reactor vessel 200. In one embodiment the acid dosage pump 414 may be activated for about 4 seconds. Priming of the acid dosage pump 414 and the acid line ensures that the quantity of acid dispensed for a first measurement will be substantially equivalent to the quantity dispensed in subsequent measurements. During priming, some acid will be delivered through the acid inlet 212 into the enclosed volume 102. This acid is able to drain through the open drain valve 236 and drain outlet 218 into the waste receptacle 458. The opening of the vent valve 454 permits air to flow through the vent and prevents a vacuum or pressurization condition from occurring in the enclosed volume 102 that may cause retention of some acid within the enclosed volume. The check valve 446 effectively holds the prime once the priming step is completed, which is one reason for locating the check valve 446 close to the acid inlet 212. Following block 508, the initialization process 500 is completed and the microprocessor 354 is directed to block 522 in FIG. 5B for performing the SO₂ measurement.

Referring to FIG. 5B, an embodiment of a measurement process is shown generally at 520. Following the initialization process 500 (or a purge process 560 described later herein) the drain valve 236 and vent valve 454 remain open. The measurement process 520 starts at block 522, which directs the microprocessor 354 to generate control signals 380 to activate the beverage dosage pump 404 and the beverage valve 410 to deliver a flush volume of beverage to the enclosed volume 102 of the reactor vessel 200. In one embodiment the flush volume may be about 4 milliliters of beverage. The flush volume when delivered to the enclosed volume 102 has the effect of flushing the enclosed volume to remove traces of residual beverage that remained in the enclosed volume after the previous measurement. The open vent valve 454 facilitates venting of gas within the enclosed volume 102 that is initially displaced by the flush volume and then allows air to flow into the enclosed volume as the flush volume is drained. Once the flush volume of beverage has been delivered and drained, the microprocessor 354 is directed to block 524.

Block 524 directs the microprocessor 354 to generate control signals 380 to deactivate the beverage dosage pump 404 and close the beverage valve 410. Block 526 then directs the microprocessor 354 to generate a control signal 380 to open the push valve 426, which permits pressurized gas from the pressurized gas supply 416 to flow through the gas line 422 and into the line connected to the liquid inlet 210 of the reactor vessel 200. Since the beverage valve 410 has already been closed at block 524, this has the effect of pushing any remaining beverage out of the liquid sample line through the liquid inlet 210, which will then be drained from the enclosed volume 102 through the drain valve 236. At this point the enclosed volume 102 has been flushed by a small amount of the beverage for which the SO₂ concentration is about to be measured, thus reducing the likelihood of cross-contamination by the prior liquid sample that had been received in the enclosed volume 102.

The measurement process 520 then continues at block 528 which directs the microprocessor 354 to start recording the signals generated by the measurement photodetector 120 and reference photodetector 334. Block 528 causes the signal values to be periodically sampled during the remainder of the measurement process 520. The resulting digital values produced by the signal conditioner 370 of the controller 350 are then received at the data input 376 and stored in the data memory 358. In one embodiment the samples are taken at a fixed sample rate and the representative values are stored in sequential memory locations for the remainder of the measurement period. For the signal conditioner 370 implemented using the Behringer audio interface the sample rate is 44.1 kHz). In this embodiment, the reference frequency U_{REF} generated by the reference frequency generator 364 is simultaneously, received at the input 368 of the I/O interface 360, converted into digital values representing the reference frequency, and stored in the data memory 358. In one embodiment the samples of the reference frequency may be taken at a fixed interval corresponding to the sample rate of the measurement and reference photodetector signals. In this embodiment the recording of the photodetector signals is commenced before the gaseous sample begins recirculating through the interior volume 112 of the flow cell 110, which provides data for balancing the measurement channel and reference channel of the optical subsystem 300 before the interior volume 112 receives the gaseous sample. A balancing process that may be executed by the processor circuit 352 after the signals have been received and stored is described later herein.

Block 530 then directs the microprocessor 354 to generate a control signal 380 to close the drain valve in preparation for receiving the liquid sample 104. The vent valve 454 remains open to allow displaced air in the enclosed volume 102 to escape through the gas outlet 214 and through the vent 456. The vented air is substantially prevented from passing into the flow cell 110 via the port 316 since the flow path through the port 318 remains blocked by the inactivated recirculation pump 450 and the check valve 452.

The measurement process 520 then continues at block 532, which directs the microprocessor 354 to generate control signals 380 to activate the beverage dosage pump 404 and to open the beverage valve 410. The beverage dosage pump 404 is operated to deliver a desired quantity of beverage through the first heat exchanger coil 440 of the heat exchanger 430 and through the liquid inlet 210 into the enclosed volume 102 of the reactor vessel 200. Block 532 also directs the microprocessor 354 to generate a control signal to activate the acid pump for delivering the quantity of acid through the second heat exchanger coil 442 of the heat exchanger 430 and through the acid inlet 212 into the enclosed volume 102. Block 534 then directs the microprocessor 354 to determine whether an acid dosage time has elapsed. In one embodiment the acid dosage pump 414 is operated for a time period sufficient to dispense a quantity of about 7.5 milliliters of acid into the enclosed volume 102. When the acid dosage time has expired block 534 directs the microprocessor 354 to block 536, which directs the microprocessor to generate control signals to deactivate the acid dosage pump 414.

Block 538 then directs the microprocessor 354 to determine whether a beverage dosage time has elapsed. In one embodiment the beverage dosage pump 404 is operated for a time period sufficient to draw a quantity of about 15 milliliters of beverage from the container 402. The beverage dosage time will thus generally extend beyond the acid dosage time. When the beverage dosage time has elapsed, block 538 directs the microprocessor 354 to block 540. Block 540 directs the microprocessor 354 to generate control signals to deactivate the beverage dosage pump 404 and to close the beverage valve 410. In this embodiment, block 540 also directs the microprocessor 354 to generate a control signal 380 to cause the push valve 426 to open to drain the liquid sample line. The pressurized gas from the pressurized gas supply 416 pushes out the remaining beverage in the liquid sample line through the liquid inlet 210 into the enclosed volume 102. In this disclosed embodiment a first portion of the quantity of beverage is thus delivered to the enclosed volume 102 by operating the liquid dosage pump 404 followed by a second portion of the quantity of beverage being delivered to the enclosed volume by flushing the liquid dosage pump using a pressurized fluid to remove residual beverage from the liquid sample line. The removal of the residual from the liquid sample line reduces the likelihood of cross-contamination with a later beverage sample drawn from a different beverage container. In other embodiments the additional step of opening the push valve 426 to drain the sample line may be omitted. Block 542 then directs the microprocessor 354 to generate a control signal to close the push valve 426. In one embodiment the push valve 426 is opened for about 4 seconds for the purpose of draining the liquid sample line. Block 542 then directs the microprocessor 354 to generate a control signal 380 to close the vent valve 454 in preparation for recirculation of the gaseous sample. At this time the optical subsystem 300 is sealed from the atmosphere to contain any released SO₂ within the headspace 106 or the interior volume 112 of the flow cell 110 and the flow lines between these volumes.

In the embodiment shown the size of the enclosed volume 102 and the quantity of beverage may be selected to fill the enclosed volume 102 such that the headspace 106 occupies a proportion of about 50% of the enclosed volume. Any entrained liquid that reaches the interior volume 112 of the flow cell 110 could deposit on the windows 310 and 312, thus potentially obstructing the optical path 118. As such, it is important to maintain separation between the liquid sample 104 and the headspace 106 to prevent liquid from being entrained in the gaseous sample drawn through the gas outlet 214 into the interior volume 112 of the flow cell 110. Fermented beverages in particular are very susceptible to foaming, which may cause liquid to be entrained in the gaseous sample. The proportion of the enclosed volume 102 occupied by the headspace 106 represents a trade-off in that a smaller headspace facilitates a more rapid release of free SO₂ from the liquid sample into the headspace, thus potentially reducing an overall measurement time. However, if the headspace 106 is too small, there is a greater risk of entrainment of liquid in the gaseous sample. In some embodiments the liquid sample 104 may occupy as much as 90% or 95% of the enclosed volume 102 and the headspace 106 proportion may thus be relatively small. In other embodiments, the liquid sample 104 may occupy as little as 15% of the enclosed volume 102. For example, the ratio of the volume of the liquid sample 104 to the headspace 106 may be 20 mL/100 mL (e.g., the liquid sample 104 may occupy 20% of the volume occupied by the headspace 106, which corresponds to 16.67% of the enclosed volume 102).

In some embodiments where foaming or liquid entrainment is not problematic the headspace proportion may be reduced below 50% of the enclosed volume 102. While in the embodiments disclosed above the gaseous sample is described as being taken from the headspace 106 and delivered to the flow cell 110, in embodiments where foaming is not problematic the UV light may be directed through the gaseous sample while in-situ within the headspace 106. In this case, the UV light may be delivered directly through the headspace 106 through windows (not shown) in the upper housing portion 202 of the reactor vessel 200. As such the separate flow cell 110 may be eliminated and effectively incorporated within the reactor vessel 200. The recirculation of gas from the headspace 106 may be maintained for purposes of sparging the liquid sample 104 or sparging may be accomplished using other mechanical or vibrational means as described above.

The process 520 then continues at block 544, which directs the microprocessor 354 to generate a control signal 380 that activates the recirculation pump 450 to draw the gaseous sample from the headspace 106 through the gas outlet 214. In this disclosed embodiment the gas conduit 222 and gas outlet 214 are at a location above the surface 108 of the liquid sample 104 and are spaced apart from the liquid sample surface to reduce the likelihood of liquid or foam entering the recirculated gaseous sample and reaching the flow cell 110. The upwardly extending passage 220 provides for further separation between the surface 108 of the liquid sample 104 and the gas outlet 214. Additionally, the gas conduit 222 being orientated at laterally (i.e. at an angle of about) 90° to the passage 220 further reduces the likelihood of any liquid or foam splashed upwardly within the enclosed volume 102 entering the gas conduit 222.

The activation of the recirculation pump 450 causes the gaseous sample to be drawn from the headspace 106 and circulated through the inlet port 316, through the interior volume 112 of the flow cell 110, and out through the outlet port 318. The recirculated gaseous sample is returned to the reactor vessel 200 through the gas inlet 216 and the sparging tube 224 for sparging the liquid sample 104. The opening 226 of the sparging tube 224 delivers the recirculated gaseous sample to the enclosed volume 102 at a location proximate a lower end of the enclosed volume 102. The opening 226 is thus directed generally downwardly and with a component directed toward a lateral wall of the enclosed volume 102, which has the effect of reducing the possibility of foaming and splashing of the liquid sample that may enter the passage 220 and gas conduit 222. Furthermore, the recirculated gaseous sample has the effect of increased mass transfer between gas phase bubbles created by the recirculated gaseous sample and the liquid sample 104 during sparging due to the increased path for the bubbles to reach the surface 108 of the liquid sample. The conditioning of the liquid sample thus involves sparging the liquid sample 104 by drawing the gaseous sample from the headspace 106 and recirculating the gaseous sample back below the surface 108 of the liquid sample to cause free SO₂ in the liquid sample to be released into the headspace.

The measurement process 520 then continues at block 546, which directs the microprocessor 354 to determine whether a recirculation time has elapsed. The recirculation should be maintained for a sufficient time to cause the free SO₂ in the liquid sample 104 to be released into the headspace 106. In one embodiment the recirculation time may be about 30 seconds.

During the recirculation time, the signals generated by the photodetectors 120 and 334 are monitored and recorded as described above in connection with block 528. When the recirculation time expires block 546 directs the microprocessor 354 to block 548. Block 548 directs the microprocessor 354 to stop recording the signals generated by the photodetectors 120 and 334. Block 548 also directs the microprocessor 354 to deactivate the recirculation pump 450, completing the measurement process 520. Block 548 then directs the microprocessor 354 to block 562 of the purge process 560 shown in FIG. 5C.

Referring to FIG. 5C, an embodiment of a purge process performed after the measurement process 520 is shown generally at 560. The purge process 560 begins at block 562, which directs the microprocessor 354 to generate control signals 380 to open the drain valve 236 and open the vent valve 454. The vent 456 permits air to enter the enclosed volume 102 through the vent valve 454, which facilitates draining the liquid sample 104 from the enclosed volume 102. Block 564 then directs the microprocessor 354 to generate control signals to open the push valve 426 and the purge valve 428. The open push valve 426 delivers the pressurized gas purge fluid 416 through the liquid inlet 210 to the enclosed volume 102. The pressurized gas forces the liquid sample 104 through the open drain valve 236 and drain outlet 218 into the waste receptacle 458. At the same time the gas line between the gas outlet 214 and the vent 456 is flushed by the pressurized gas through the vent 456. The open purge valve 428 also delivers pressurized gas via the outlet port 318 to the outlet port 318 of the flow cell 110. Since the recirculation pump 450 is not activated the flow is effectively blocked by the pump. The pressurized gas thus flows through the outlet port 318, purges the interior volume 112 of the flow cell 110, and is vented through the vent 456.

Block 566 then directs the microprocessor 354 to determine whether the purge time has expired. In one embodiment the purge time may be about 5 seconds. When the purge time has expired, the microprocessor 354 is directed to block 568, which directs the microprocessor to generate control signals 380 to cause the vent valve 454 to close. When the vent valve 454 closes, a full flow of pressurized gas being delivered through the liquid inlet 210 and via the purge valve 428 through the flow cell 110 will be directed through the drain valve 236, which causes a surge to drive a substantial remaining portion of the liquid sample 104 through the drain valve.

The purge process 560 then continues at block 570, which directs the microprocessor 354 to generate control signals 380 to reverse the beverage dosage pump 404 and to activate the recirculation pump 450. The reversing of the beverage dosage pump 404 draws pressurized gas through the beverage valve 410 which purges the liquid sample line 406 and dip tube 408. The recirculation pump 450 also draws the pressurized gas through the gas line 424 and purge valve 428 to flush the recirculation pump 450 and the gas inlet 216 with the pressurized gas. The pressurized gas vents through the drain valve 236. Block 572 then directs the microprocessor 354 to generate control signals 380 to deactivate the recirculation pump and beverage dosage pump.

Following completion of the purge process 560, the enclosed volume 102 of the reactor vessel, interior volume 112 of the flow cell, the sparging tube 224, the gas lines, liquid sample line, and the pumps 404 and 450 will all have all been flushed by the pressurized gas. The use of an inert gas such as nitrogen as the pressurized gas would effectively remove a substantial portion of the liquid sample 104 and beverage sample from the monitoring system 400 and reduce the likelihood of cross-contamination occurring in a subsequent SO₂ measurement performed for a different beverage. The drain valve 236 and vent valve 454 may remain open to prepare the beverage monitoring system 400 for a subsequent measurement, since the measurement process 520 starts out with these valves in this state.

The reference channel of the optical subsystem 300 may be used by the controller 350 to correct for any noise in the UV light beam 322 generated by the UV light source 116. Small concentrations of SO₂ in the gaseous sample will typically result in small signal differences that can easily be obscured in the noise due to the UV light source 116 or other elements of the optical subsystem 300 and controller 350. The reference channel and measurement channel would generally be subjected to the same noise sources and the reference signal may thus be used to attenuate the noise, thus increasing the signal to noise ratio for discriminating absorption effects due to SO₂ on the measurement signal produced by the measurement photodetector 120.

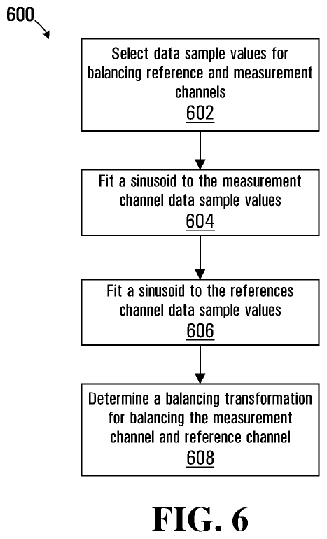

Referring to FIG. 6, a channel balancing process is shown generally at 600. The process begins at block 602, which directs the microprocessor 354 to select data sample values form the recorded photodetector signals for both the reference channel and measurement channel. These samples would typically be selected from the first samples recorded starting at block 568 of the measurement process 520 during a time tᵢ. At this time, the flow cell 110 remains in a purged state established by executing the purge process 560 after a previous measurement on a beverage had been completed. The signal level produced by the measurement photodetector 120 will thus be effectively unattenuated by its passage through the flow cell 110 since the interior volume 112 would have been flushed of SO₂ from the previous measurement by the purge fluid 416.

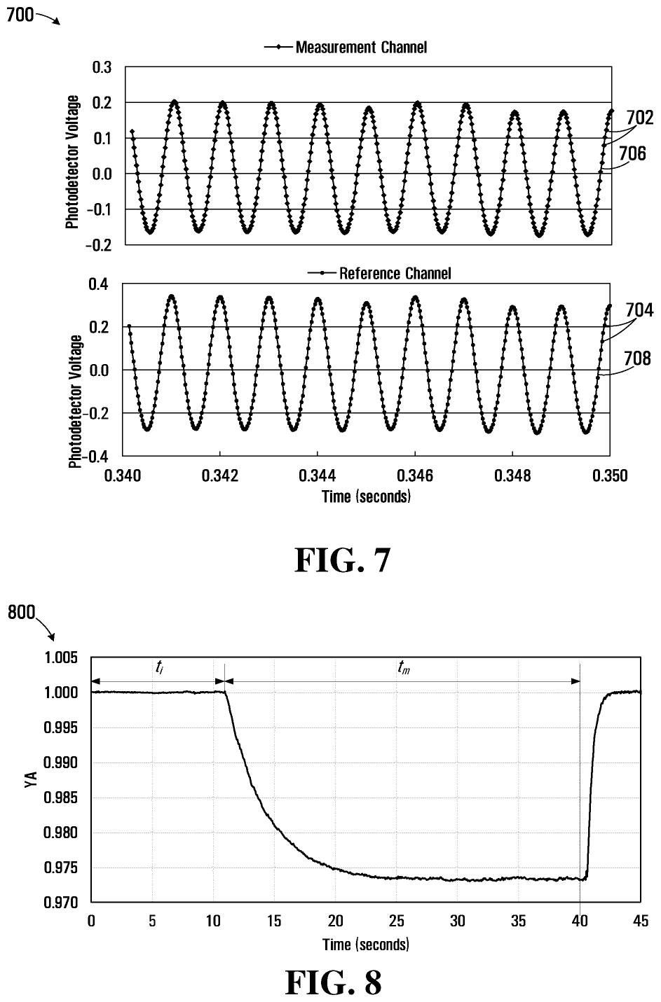

Examples of signals generated by the measurement photodetector 120 and the reference photodetector 334 during the initial time t; while the quantity of beverage is being delivered to the enclosed volume 102 are shown graphically in FIG. 7 at 700. Referring to FIG. 7, the measurement channel signal produced by the measurement photodetector 120 is shown at 702. In this embodiment the UV light source 116 is sinusoidally modulated at about 1000 Hz and the syMbols “♦” represent data sample values that are generated by the signal conditioner 370 of the controller 350 in response to receiving the signal produced by the measurement photodetector 120. The reference channel signal produced by the reference photodetector 334 is shown at 704 and the syMbols “•” represent data sample values that are generated by the signal conditioner 370 of the controller 350 in response to receiving the signal produced by the reference photodetector. Each data sample value is thus represented by a pair of values (y,t) including a photodetector voltage y and a sample time t. In this embodiment the data sample values 702 from the measurement channel are at a slightly higher level than the data sample values 704 from the reference channel due to a small imbalance between the channels.

The process then continues at block 604, which directs the microprocessor 354 to fit a sinusoid to the data sample values 702 for the measurement channel. In one embodiment a linear regression may be performed using the following function:

$$y ( t ) = a sin ( 2 πft ) + b cos ( 2 πft ) + c . \qquad Eqn 1$$

Each data sample value y at a sample time t in the data sample values 702 may be substituted into Eqn 1 above to perform a linear regression that yields the parameters a, b, and c. The determined parameters a, b, and c may be used to define a sinusoid 706 for the measurement channel, written as:

$${y}_{M} = {A}_{M} sin ( 2 πft + {ϕ}_{M} ) + {c}_{M} , \qquad Eqn 2$$

where:
y_{M} is the measured signal;
A_{M} is the amplitude of the sinusoid;
f is the frequency in Hz;
t is the time in seconds;
ϕ_{M} is the phase shift of the sinusoid; and
C_{M} is the dc offset of the photodetector signal (0.080 V).

The process then continues at block 606, which directs the microprocessor 354 to repeat the above linear regression for the data samples to fit a sinusoid to the data sample values 704 for the reference channel:

$${y}_{R} = {A}_{R} sin ( 2 πft + {ϕ}_{R} ) + {c}_{R} , \qquad Eqn 3$$

where:
y_{R} is the reference signal;
A_{R} is the amplitude of the sinusoid;
ϕ_{R} is the phase shift of the sinusoid; and
C_{R} is the DC offset of the photodetector signal.

Even if the measurement photodetector 120 and reference photodetector 334 are matched and the beam-splitter 324 is configured for a 50:50 split ratio, there may be slightly different gain and/or DC offset between the measurement and reference channels that cause the sinusoids 706 and 708 to be separated on the y axis and also have slightly differing amplitude A_{M} and A_{R}.

Block 608 then directs the microprocessor 354 to determine a balancing transformation for balancing the reference channel to the measurement channel. The balancing transformation may be applied to subsequent reference channel data sample values y_{R} to provide a balanced reference signal y_{RB}:

$${y}_{RB} = [ \frac{{A}_{M}}{{A}_{R}} [ {y}_{R} ( t - {ϕ}_{R} + {ϕ}_{M} ) - {c}_{R} ] + {c}_{M} ] . \qquad Eqn 4$$

In practice, the phase shifts ØR and Om may be considered negligible for the optical subsystem 300 shown in FIG. 3 and may be ignored.

In the embodiment described above the UV light generated by the UV light source 116 is sinusoidally modulated in intensity. In other embodiments various other modulation techniques that place a modulation signature on the UV light, such as wavelength modulation, may be implemented with similar effect.

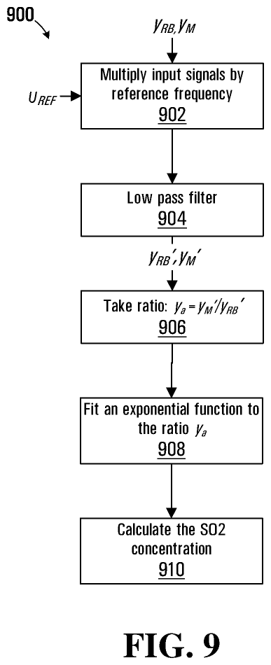

In one embodiment synchronous detection (lock-in amplification) may be performed on the reference signal y_{RB} and measurement signal y_{M}. The lock-in amplification further improves the signal to noise ratio of the absorption and reference signals. Referring to FIG. 9, a digital signal process executed by the processor circuit 352 in accordance with one disclosed embodiment is shown generally at 900. The process 900 is separately executed for each of the signals y_{M} and y_{RB}. Block 902 directs the microprocessor 354 to multiply each of the signals y_{M} and y_{RB} by the reference frequency U_{REF}. The resulting signals thus become:

$${y}_{M}^{′} = \frac{1}{2} {y}_{M} {v}_{REF} [ cos ( [ 2 π ( {f}_{M} - {f}_{REF} ) ] t + {ϕ}_{M} - {ϕ}_{REF} ) - cos ( [ 2 π ( {f}_{M} + {f}_{REF} ) ] t + {ϕ}_{M} + {ϕ}_{REF} ) ] \qquad Eqn 5$$

$$and$$

$${y}_{RB}^{′} = \frac{1}{2} {y}_{RB} {v}_{REF} [ cos ( [ 2 π ( {f}_{RB} - {f}_{REF} ) ] t + {ϕ}_{RB} - {ϕ}_{REF} ) - cos ( [ 2 π ( {f}_{RB} + {f}_{REF} ) ] t + {ϕ}_{RB} + {ϕ}_{REF} ) ] \qquad Eqn 6$$

where:
f_{M} is the frequency of the measured signal y_{M};
f_{RB} is the frequency of the balanced reference signal y_{RB};
f_{REF} is the frequency of the reference frequency U_{REF};
ϕ_{M} is the phase of the measured signal y_{M};
ϕ_{RB} is the phase of the reference signal y_{RB}; and
ϕ_{REF} is the phase of the reference frequency U_{REF}.

The output signals in equations 5 and 6 thus each have two AC components, one at a difference frequency (f_{M}−F_{REF}) or (F_{RB}−f_{REF}) and the other at a sum frequency (f_{M}+f_{REF}) or (F_{RB}+f_{REF}). The process then continues at block 904, which directs the microprocessor 354 to low pass filter the signals y′_{M} and y′_{RB} to remove the sum frequency component in equations 5 and 6. Additionally, since the frequency f_{M} and f_{RB} are both derived from the reference frequency f_{REF} generated by the reference frequency generator 364 the difference frequencies (f_{M}−f_{REF}) and (f_{RB}−f_{REF}) will both be zero. For the optical subsystem 300 the phase shifts should also be negligible and thus the remaining cosine term in equations 5 and 6 will be unity and the output signals will be:

$${y}_{M}^{′} \approx \frac{1}{2} {y}_{M} {v}_{REF} \qquad Eqn 7$$

$$and$$

$${y}_{RB}^{′} = \frac{1}{2} {y}_{RB} {v}_{REF} , \qquad Eqn 8$$

which are both DC signals proportional to the amplitude of y_{M} and y_{RB}.

In the digital lock-in amplifier implementation shown in FIG. 9, the signals and reference frequency are both represented by sequences of numbers and the multiplication and filtering are performed mathematically by using digital signal processing functions.

Block 906 of the process 900 then directs the microprocessor 354 to further process the signals y′M and y_{RB} to yield an output absorption signal y_{A} by taking a ratio of the data sample values:

$${y}_{A} = \frac{{y}_{M}^{′}}{{y}_{RB}^{′}} . \qquad Eqn 9$$

A graphical depiction of y_{A} is shown graphically in FIG. 8 at 800. As disclosed above in connection with block 528 of the measurement process 520, recording of the signals generated by the photodetectors 120 and 334 begins after the enclosed volume 102 has been flushed with a quantity of the beverage for which the SO₂ concentration is being determined. The recording of the signals generated by the photodetectors 120 and 334 continues through the measurement process 520 up to block 548, which directs the microprocessor 354 to stop recording the signals. During the initial time period t; the processed signal yₐ has a value close to unity due to the above ratio being taken between the measurement signal values and the reference signal values, which have been balanced by the channel balancing process 600. During the time tᵢ, the measurement channel has negligible absorption since the gaseous sample has not yet been introduced into the interior volume 112 of the flow cell 110. Toward the end of the time period t; the quantity of beverage is introduced into the enclosed volume 102.

During a second time period tₘ the quantity of acid is introduced into the enclosed volume 102, which will promote the release of free SO₂ from the liquid sample 104 into the headspace 106 through acid hydrolysis. The recirculation pump 450 is also activated and the gaseous sample will thus begin to be delivered to the interior volume 112 of the flow cell 110. The SO₂ concentration in the interior volume 112 of the flow cell 110 will begin to increase due to the combination of the sparging action and acid hydrolysis. The SO₂ concentration will thus start to increase causing a corresponding increase in absorption and thus a decrease in the ratio signal yₐ. At some point during the time tₘ, the SO₂ concentration within the interior volume 112 reaches an equilibrium and the signal yₐ thus approaches an asymptote. At the end of the time tₘ recording of the signal generated by the measurement photodetector 120 may be discontinued.

The process 900 then continues at block 908, which directs the microprocessor 354 to fit an exponential function to the absorption ratio signal yₐ values that fall within the time period tₘ. The optical absorption due to a gas such as SO₂ is given by the Beer-Lambert law, which may be written in the form:

$$A = \frac{{P}_{O}}{P} = {e}^{ϵ bC} , \qquad Eqn 10$$

where:
A is the absorption within the gaseous sample due to a concentration C of SO₂;
E is the molar absorption coefficient for SO₂; and
b is the length of the optical path 118.

Fitting an exponential function to the yₐ values during the time tₘ thus yields a value for the exponent εbC. In other embodiments, another function may be fitted to the yₐ values to yield a value for εbC. For example, a function with a larger number of degrees of freedom may be used. Block 910 then directs the microprocessor 354 to calculate the SO₂ concentration C using known values of the molar absorption coefficient e and the length of the optical path 118.

The above disclosed embodiments provide for a rapid and accurate determination of SO₂ concentration in the presence of CO₂. In one embodiment the elapsed time between executing block 528 and executing block 548 may be about 50 seconds. The purge process 560 adds about 10 seconds for a total measurement time of about 1 minute. This test time makes the SO₂ measurement system 100 particularly suitable for applications where SO₂ monitoring is to be done on a large number of beverage containers, such as in a winery using wooden barrels. The implementation of various anti-foaming features in both the reactor vessel and the measurement process reduces the possibility of liquid fouling of the optical subsystem 300, which generally would necessitate cleaning downtime.

While specific embodiments have been described and illustrated, such embodiments should be considered illustrative only and not as limiting the disclosed embodiments as construed in accordance with the accompanying claims.

What is claimed is:

1. A method for rapid determination of a concentration of free sulfur dioxide (SO₂) in a liquid, the method comprising:
receiving a liquid sample in an enclosed volume, the liquid sample including:
a quantity of the liquid; and
a quantity of acid to promote gasification of SO₂ within the liquid;
while a temperature of the liquid sample remains below 35° C., conditioning the liquid sample to cause gasified SO₂ to accumulate in a headspace of the enclosed volume above a surface of the liquid sample to provide a gaseous sample;
directing UV light through the gaseous sample, the UV light having a wavelength within a spectral range of between 250 nm and 320 nm;
measuring an attenuation of the UV light due to absorption within the gaseous sample; and
determining the concentration of gaseous SO₂ within the gaseous sample from the measured attenuation, the gaseous SO₂ concentration being indicative of the free SO₂ concentration within the liquid sample.

2. The method of claim 1 wherein the liquid comprises a beverage.

3. The method of claim 1 wherein the liquid includes dissolved or entrained carbon dioxide (CO₂) and wherein conditioning the liquid sample comprises causing at least some CO₂ within the liquid sample to accumulate in the headspace, and wherein determining the attenuation of the UV light comprises determining the attenuation of the UV light in the presence of CO₂ in the gaseous sample.

4. The method of claim 1 further comprising causing the liquid sample to have a temperature within a temperature range of between 18° C. and 35° C.

5. The method of claim 4 wherein receiving the liquid sample comprises passing the liquid sample through a heat exchanger to heat the liquid sample to a temperature within the temperature range.

6. The method of claim 1 further comprising drawing the gaseous sample from the headspace and delivering the gaseous sample to a flow cell and wherein directing the UV light through the gaseous sample comprises directing UV light through the flow cell.

7. The method of claim 1 wherein conditioning the liquid sample comprises sparging the liquid sample by drawing the gaseous sample from the headspace and recirculating the gaseous sample back below the surface of the liquid sample to cause free SO₂ in the liquid sample to be released into the headspace.

8. The method of claim 7 wherein sparging the liquid sample comprises delivering the recirculated gaseous sample to the enclosed volume at a location proximate a lower end of the enclosed volume.

9. The method of claim 8 wherein delivering the recirculated gaseous sample comprises directing the recirculated gaseous sample generally downwardly in the enclosed volume and with a component directed toward a lateral wall of the enclosed volume.

10. The method of claim 7 wherein recirculating the gaseous sample comprises passing the gaseous sample through a flow cell having an optical path therethrough, and wherein directing UV light through a gaseous sample comprises directing UV light through the optical path of the flow cell.

11. The method of claim 10 wherein drawing the gaseous sample comprises drawing the gaseous sample from a location above and spaced apart from the surface of the liquid sample to reduce a likelihood of liquid or foam from the liquid sample entering the recirculated gaseous sample and reaching the flow cell.

12. The method of claim 11 wherein the enclosed volume comprises a passage that extends the headspace of the enclosed volume upwardly away from the surface of the liquid sample and wherein drawing the gaseous sample comprises drawing the gaseous sample from a location proximate an upper end of the passage.

13. The method of claim 12 wherein drawing the gaseous sample comprises drawing the gaseous sample from the passage in a lateral direction.

14. The method of claim 1 wherein receiving the liquid sample comprises causing a liquid dosage system to draw the quantity of liquid from a container, and wherein:
a first portion of the quantity of liquid is delivered to the enclosed volume by operating the liquid dosage system; and
a second portion of the quantity of liquid is delivered to the enclosed volume by flushing the liquid dosage system using a pressurized fluid.

15. The method of claim 14 further comprising opening a vent valve in fluid communication with the headspace of the enclosed volume while flushing the liquid dosage system to permit the pressurized fluid to escape.

16. The method of claim 1 wherein the enclosed volume comprises a drain port sealed by a drain valve at the bottom of the enclosed volume and wherein the method further comprises, after the concentration of gaseous SO₂ within the gaseous sample has been determined, causing the drain valve to open to permit the liquid sample to be drained from the enclosed volume.

17. The method of claim 16 wherein the drain valve has a frustoconical shape and is received in a frustoconical valve seat at the bottom of the enclosed volume and wherein the valve is configured to open by lifting upwardly out of the valve seat such that when being drained the liquid sample is completely drained to the bottom of the enclosed volume.

18. The method of claim 16 further comprising flushing the enclosed volume by delivering a fluid to the enclosed volume that causes any remnants of the liquid sample to be forced out of the drain.

19. The method of claim 1 wherein directing UV light comprises directing UV light produced by a UV light emitting diode through the gaseous sample.

20. The method of claim 1 wherein measuring the attenuation of the UV light comprises:
generating modulated UV light;
generating a measurement signal in response to receiving the UV light at a photodetector after passing through the gaseous sample;
processing the measurement signal to extract components that are synchronized with the modulated UV light to determine an attenuation of the UV light; and
determining the attenuation by comparing the level of attenuated UV light with a level of the generated modulated UV light.

21. The method of claim 20 wherein generating modulated light comprises generating UV light that is intensity modulated at a reference frequency and wherein processing the measurement signal comprises processing the measurement signal to extract components that are synchronized with the reference frequency to determine the attenuation of the UV light.

22. The method of claim 20 wherein the photodetector comprises a silicon carbide photodetector that is responsive to wavelengths of UV light in a narrow band including the 280 nanometer wavelength of the UV light.

23. The method of claim 20 further comprising converting the measurement signal produced by the photodetector into a digital representation for receipt by a processor circuit and wherein processing the measurement signal comprises mathematically processing the measurement signal in the processor circuit.

24. The method of claim 20 further comprising splitting the UV light into a first beam of UV light and a second beam of UV light and wherein directing UV light through the gaseous sample comprises directing the first beam of UV light along a measurement optical path through the gaseous sample in a measurement channel and further comprising directing the second beam of UV light through a reference optical path in a reference channel to generate a reference signal for extracting the measurement signal from noise.

25. The method of claim 24 further comprising balancing the measurement channel and the reference channel while no gaseous sample is present.

26. The method of claim 20 wherein determining the concentration of gaseous SO₂ comprises:
producing an output absorption signal by taking a ratio of the measurement signal values and the reference signal; and
determining the SO₂ concentration based on fitting an exponential function to the output absorption signal.

27. The method of claim 1 wherein the quantity of acid comprises a quantity of phosphoric acid to lower a pH of the liquid sample to below pH 3.

28. A system for rapid determination of a concentration of free sulfur dioxide (SO₂) in a liquid, the system comprising:
an enclosed volume operable to receive a liquid sample, the liquid sample including:
a quantity of the liquid; and
a quantity of acid to promote gasification of SO₂ within the liquid;
a liquid sample conditioner operably configured to condition the liquid sample to cause gasified SO₂ to accumulate in a headspace of the enclosed volume above a surface of the liquid sample while a temperature of the liquid sample remains below 35° C.;
a flow cell in fluid communication with the headspace of the enclosed volume, the flow cell being operable to receive a gaseous sample taken from the headspace of the enclosed volume, the flow cell having an optical path therethrough;
a UV light source having a wavelength within a spectral range of between 250 nm and 320 nm, the UV light source being disposed to direct UV light through the optical path of the flow cell;
a photodetector disposed to receive UV light passing through the gaseous sample and to generate a measurement signal representing an attenuation of the UV light due to absorption within the gaseous sample; and
a processor operably configured to determine the concentration of gaseous SO₂ within the gaseous sample based on the attenuation signal, the gaseous SO₂ concentration being indicative of the free SO₂ concentration within the liquid sample.

29. The system of claim 28 wherein the liquid comprises a beverage.
